SEQUENCE LISTING (29 sequences)

SEQ ID NO: 1 - DNA, 25 bp, Mus musculus
gctaccaaactggatataatcagga

SEQ ID NO: 2 - DNA, 24 bp, Mus musculus
ccaggtagctatggtactccagaa

SEQ ID NO: 3 - DNA, 20 bp, Mus musculus
gattcagactccaggggaca

SEQ ID NO: 4 - DNA, 23 bp, Mus musculus
tggttagcttctgaggacacatc

SEQ ID NO: 5 - DNA, 24 bp, Mus musculus
ccatcagcagatcattctagacaa

SEQ ID NO: 6 - DNA, 22 bp, Mus musculus
cgccattatgattcagagactg

SEQ ID NO: 7 - DNA, 21 bp, Mus musculus
gctgttgatggacctacagga

SEQ ID NO: 8 - DNA, 20 bp, Mus musculus
ttcaattctgtggcctgctt

SEQ ID NO: 9 - DNA, 19 bp, Mus musculus
catcggcattttgaacgag

SEQ ID NO: 10 - DNA, 20 bp, Mus musculus
cgagctcactctctgtggtg

SEQ ID NO: 11 - DNA, 20 bp, Mus musculus
atctggaggaactggcaaaa

SEQ ID NO: 12 - DNA, 26 bp, Mus musculus
ttcaagacttcaaagagtctgaggta

SEQ ID NO: 13 - DNA, 21 bp, Mus musculus
tcttctcattcctgcttgtgg

SEQ ID NO: 14 - DNA, 20 bp, Mus musculus
ggtctgggccatagaactga

SEQ ID NO: 15 - DNA, 21 bp, Mus musculus
cagggagagcttcatctgtgt

SEQ ID NO: 16 - DNA, 20 bp, Mus musculus
gctgagctttgagggatgat

SEQ ID NO: 17 - DNA, 22 bp, Mus musculus
tccctactaggactcagccaac

SEQ ID NO: 18 - DNA, 18 bp, Mus musculus
tgggcatctgttgggtct

SEQ ID NO: 19 - DNA, 18 bp, Mus musculus
gggctgtcacggagatca

SEQ ID NO: 20 - DNA, 20 bp, Mus musculus
ccatgatggtcacattctgc

SEQ ID NO: 21 - DNA, 20 bp, Mus musculus
cagagccacatgctcctaga

SEQ ID NO: 22 - DNA, 20 bp, Mus musculus
gtccagctggtcctttgttt

SEQ ID NO: 23 - DNA, 23 bp, Mus musculus
cctctgacccttaaggagcttat

SEQ ID NO: 24 - DNA, 18 bp, Mus musculus
cgttgcacaggggagtct

SEQ ID NO: 25 - protein, 21 aa, Mus musculus
MEVGWYRSPFSRVVHLYRNGK

SEQ ID NO: 26 - protein, 147 aa, Homo sapiens [sequence omitted]

SEQ ID NO: 27 - protein, 143 aa, Homo sapiens
MSQTRDLQGGKAFGLLKAQQEERLDEINKQFLHDPKYSSDEDLPSKLEGFKEKYMEFDLNGNGDIDIMSLKRMLEKLGVPKTHLELKKLIGEVSSGSGETFSYPDFLRMMLGKRSAILKMILMYEEKARERKTNTPPSQESPI

SEQ ID NO: 28 - protein, 132 aa, Homo sapiens
MEFDLNGNGDIGEKRVICGGRVVCRPKKTEVSPTCSIPHDLGGGPPTTVGGRRMGMRKWERRERVSPPSPHPHPLPPDIMSLKRMLEKLGVPKTHLELKKLIGEVSSGSGETFSYPDFLRMMLGKRSAILKM

SEQ ID NO: 29 - protein, 93 aa, Homo sapiens
MEFDLNGNGDIDIMSLKRMLEKLGVPKTHLELKKLIGEVSSGSGETFSYPDFLRMMLGKRSAILKMILMYEEKAREKEKPTGPPAKKAISELP

(19) United States
(12) Patent Application Publication
(10) Pub. No.: US 2017/0073677 A1
(43) Pub. Date: Mar. 16, 2017

(54) TREATMENT OF MULTIPLE SCLEROSIS BY INHIBITION OF ALLOGRAFT INFLAMMATORY FACTOR-1

(71) Applicant: ALBERT EINSTEIN COLLEGE OF MEDICINE, INC., Bronx, NY (US)

(72) Inventors: Nicholas Ernst Smit SIBINGA, Chappaqua, NY (US); Prameladevi CHINNASAMY, Bronx, NY (US); Sarah E. LUTZ, Irvine, CA (US)

(73) Assignee: ALBERT EINSTEIN COLLEGE OF MEDICINE, INC., Bronx, NY (US)

(21) Appl. No.: 15/308,105

(22) PCT Filed: May 6, 2015
(86) PCT No.: PCT/US2015/029344

§ 371 Date: Nov. 1, 2016

Related U.S. Application Data
(60) Provisional application No. 62/000,577, filed on May 20, 2014.

(51) Int. Cl. C12N 15/113; G01N 33/50

(52) U.S. Cl. CPC C12N 15/113; G01N 33/5047; C12N 2320/30; G01N 2333/4727; G01N 2800/285

(57) ABSTRACT

Methods are disclosed for treating multiple sclerosis comprising administering an agent that reduces expression and/or activity of Allograft inflammatory factor-1 (Aif-1) in a subject and for screening for such agents.

### CROSS-REFERENCE TO RELATED APPLICATIONS

This application claims the benefit of U.S. Provisional Patent Application No. 62/000,577, filed May 20, 2014, the contents of which are herein incorporated by reference in their entirety.

### STATEMENT OF GOVERNMENT SUPPORT

This invention was made with government support under grant number HL67944 awarded by the National Institutes of Health. The government has certain rights in the invention.

### BACKGROUND OF THE INVENTION

Throughout this application various publications are referred to in parentheses. Full citations for these references may be found at the end of the specification. The disclosures of these publications are hereby incorporated by reference in their entirety into the subject application to more fully describe the art to which the subject invention pertains.

Multiple sclerosis (MS) is a chronic progressive disorder caused by the formation of inflammatory plaques in brain and spinal cord (1). MS affects about 400,000 people in the United States, and the World Health Organization estimates that between 2 and 2.5 million people are affected globally. The disease usually begins between the ages of 20 and 50 and is twice as common in women as men. Studies of MS patients and experimental autoimmune encephalomyelitis (EAE), an animal model for MS, provide convincing evidence that T lymphocytes specific for myelin antigens mediate pathology in these diseases (2). EAE shares both neuro-pathological and clinical features of MS (3). EAE can be induced by immunization with spinal cord homogenates or various myelin-associated proteins, or by adoptive transfer of antigen (Ag)-sensitized T lymphocytes from immunized animals. The inflammatory response in EAE is mediated by MHC class II-restricted, Th1-type CD4+ myelin reactive and Th17-type T cells (4-6). Auto-reactive T cells are activated in the periphery, cross the blood brain barrier, and enter the CNS. These self-reactive T cells are important initiators of the disease, controlling subsequent recruitment and activation of various effector cells. Pathogenic T cells and their pro-inflammatory cytokine milieu drive the inflammatory processes of EAE in both humans and mice (7-9). Microglia and macrophages also actively participate in EAE pathogenesis in complex ways, both through cytokine production that exacerbates inflammation during induction, and through phagocytic activities that clear cell apoptotic bodies, debris, and inhibitory substances that limit remyelination and axon regeneration (10, 11). Microglia may be important for neuro repair functions (10, 11).

Allograft inflammatory factor-1 (Aif-1, also known as ionized Ca2+ binding adapter-1 (Iba-1)) is a 17 kDa, IFN-γ-inducible, EF hand motif protein encoded within the class III region of the MHC (human chromosome 6p21.3, mouse chromosome 17B1) in an area densely clustered with inflammatory response genes, including those encoding TNF, lymphotoxin-α and -β, and components of the complement cascade (12, 13). Largely similar gene products arising from the same locus have been named Iba1, microglial response factor-1 (MRF1), and daintain; Iba1 in particular is well known as a histologic marker of microglia and of their activation in pathologic CNS conditions. Aif-1 is differentially expressed in various mouse and human tissues such as thymus, spleen, liver, brain, and testis (14, 15) and in multiple leukocyte types including macrophages, T cells, and peripheral blood mononuclear cells at basal levels (16-18). In inflammatory disease models, upregulated Aif-1 expression has been identified in microglia, macrophages, T cells, synoviocytes, pancreatic β-cells, and adipocytes under various pathologic conditions representing encephalomyelitis, uveitis, neuritis, arteriopathies, arthritis, diabetes, and obesity, respectively (19, 20).

Despite heightened Aif-1 expression in various inflammatory conditions (21, 22), its functional significance in diseases such as MS and EAE remains unknown. In the MOLT-4 T cell line, Aif-1 overexpression in vitro increases proliferation, migration, and activation (17), while its overexpression in macrophage cell lines leads to increased production of IL-6, IL-12, and IL-10 after lipopolysaccharide stimulation (23). On the other hand, impaired Aif-1 function decreases microglial phagocytosis (24). These in vitro findings suggest that Aif-1 deficiency in EAE could be beneficial, due to decreased pro-inflammatory activities of T cells and macrophages, but on the other hand could also impair phagocytosis, allowing cellular debris to accumulate and secondarily promoting inflammation and neurotoxicity and impairing regenerative processes.

The present invention addresses the need for improved treatments for multiple sclerosis.

### SUMMARY OF THE INVENTION

The present invention provides methods of treating multiple sclerosis in a subject comprising administering to the subject an agent in an amount effective to reduce expression and/or activity of Allograft inflammatory factor-1 (Aif-1) in a subject.

The invention further provides methods for screening for an agent that treats multiple sclerosis in a subject, the methods comprising determining whether or not the agent reduces expression and/or activity of Allograft inflammatory factor-1 (Aif-1), wherein an agent that reduces expression and/or activity of Aif-1 is a candidate for treating multiple sclerosis.

### BRIEF DESCRIPTION OF THE DRAWINGS

FIG. 1A. Aif-1 inactivation limits EAE in mice. WT and aif-1−/− mice were sensitized with MOG35-55 and evaluated. Clinical scoring of EAE activity in WT (upper trace) and aif-1−/− mice (lower trace); data were pooled from two independent experiments and shown as mean±SEM (n=16 (WT); n=13 (aif-1−/−). The P values refer to comparison between WT and aif-1−/− mice. *, P<0.05, **, P<0.01.

FIG. 1B. Aif-1 inactivation limits EAE in mice. Clinical expression was quantified by measuring the area under curve (AUC) in WT and aif-1−/− mice. The P values refer to comparison between WT and aif-1−/− mice. *, P<0.05.

FIG. 1C. Aif-1 inactivation limits EAE in mice. Quantification of mononuclear cell infiltration in the submeningeal areas of lumbar spinal cord from day 16 EAE lesions in WT and aif-1−/− mice. Sections were stained by H&E, and infiltration was quantified using Adobe Photoshop image analysis and expressed as a percentage of the total area, as described in Methods (n=4 per group). The P values refer to comparison between WT and aif-1−/− mice. *, P<0.05.

FIG. 1D. Aif-1 inactivation limits EAE in mice. Quantification of demyelination in lumbar spinal cords from day 16 EAE lesions in WT and aif-1−/− mice (n=4 per group). Sections were stained with antibody against Myelin basic protein and with DAPI nuclear stain. The area of demyelination was quantified using Adobe Photoshop image analysis and expressed as a percent of the total area, as described in Methods. The P values refer to comparison between WT and aif-1−/− mice. *, P<0.05.

FIG. 2A. Aif-1−/− mice showed decreased immune cell infiltration into CNS. Representative FACS data of effector T cells (CD45+CD3+CD4+; CD45+CD3+CD8+), microglia (CD45lowCD11b+) and infiltrated monocytes/activated microglia (CD45highCD11b+).

FIG. 2B. Aif-1−/− mice showed decreased immune cell infiltration into CNS. Infiltrated leukocytes and microglia from 13 mice per genotype were characterized and quantified by FACS, with results pooled from two independent experiments. Data are represented as mean±SEM. *, P<0.05, **, P<0.01. Dark shading—WT; Lighter shading—aif-1^{-/-}.

FIG. 3A. Aif-1 deficiency reduces CD4 T cell expansion and activation in the spleen. Splenocytes were collected from day 16 EAE and analyzed by FACS to quantify B cells (CD3−B220+), effector T cells (CD3+CD4+; CD3+CD8+) and monocytes (CD45+CD11b+) from WT and aif-1−/− mice. Mean percentage of respective population (n=6 per group). Data are represented as mean±SEM. *, P<0.05. Dark shading—WT; Lighter shading—aif-1^{-/-}.

FIG. 3B. Aif-1 deficiency reduces CD4 T cell expansion and activation in the spleen. T cell proliferation was measured by rechallenging splenocytes with α-CD3 to measure antigen specific proliferation using [3H]-thymidine incorporation (n=6 per group). Data are represented as mean±SEM. *, P<0.05. Dark shading—WT; Lighter shading—aif-1^{-/-}.

FIG. 3C. Aif-1 deficiency reduces CD4 T cell expansion and activation in the spleen. T cell proliferation was measured by rechallenging splenocytes with MOG35-55 to measure antigen specific proliferation using [3H]-thymidine incorporation (n=6 per group). Data are represented as mean±SEM. ****, P<0.0001. Dark shading—WT; Lighter shading—aif-1^{-/-}.

FIG. 3D. Aif-1 deficiency reduces CD4 T cell expansion and activation in the spleen. Data of activated T cell subsets (CD4+CD69+; CD8+CD69+ of splenocytes from WT and aif-1−/− mice isolated from day 16 EAE. Mean percentage of CD4 and CD69 activation (n=6 per group) are shown. Data are represented as mean±SEM. *, P<0.05. Dark shading—WT; Lighter shading—aif-1^{-/-}.

FIG. 4A. Relative mRNA expression of cytokines measured from day 16 EAE spleens of WT and aif-1−/− mice, normalized to gapdh (n=7). Data are represented as mean±SEM. *, P<0.05, **, P<0.01. Dark shading—WT; Lighter shading—aif-1−/−.

FIG. 4B. Protein levels of cytokines (IL-6, IFN-γ, IL-2 and IL-12p40) after rechallenge with MOG35-55 measured by ELISA (n=4 per group). Data are represented as mean±SEM. *, P<0.05. Dark shading—WT; Lighter shading—aif-1−/−.

FIG. 5. Inhibition of Aif-1 expression by specific short interfering RNA (siRNA). RAW264.7 macrophages were transfected with non-targeting (control) or aif-1-specific siRNAs. Total cellular lysates (25 μg per lane, in triplicate) were harvested after 48 h and Aif-1 protein levels were evaluated by Western analysis. Gapdh protein is shown as a loading control.

FIG. 6. Aif-1 potentiates NFkB activation. RAW264.7 macrophages were transfected with a control expression vector or Aif-1-encoding expression vector, and stimulated with interferon-gamma (100 ρ/ml) and LP S (5 ng/ml). After 8 h, luciferase activity was assessed.

### DETAILED DESCRIPTION OF THE INVENTION

The present invention provides a method of treating multiple sclerosis in a subject comprising administering to the subject an agent in an amount effective to reduce expression and/or activity of Allograft inflammatory factor-1 (Aif-1) in a subject.

As used herein, to treat a subject with multiple sclerosis means to ameliorate a sign or symptom of multiple sclerosis. Signs and symptoms of multiple sclerosis include for example, but are not limited to, neurological, autonomic, visual, motor, and sensory signs and symptoms.

Aif-1 expression and/or activity can be reduced, for example, in lymphocytes and/or in macrophages and/or microglia.

In one embodiment of the methods described herein, the agent is an antisense molecule, a ribozyme, or an RNA interference (RNAi) molecule, such as short interference RNA (siRNA) (e.g., 38) or short hairpin RNA (shRNA), where the antisense molecule, ribozyme or RNAi molecule specifically reduces expression of Aif-1. The antisense molecule, ribozyme, or RNAi molecule can be comprised of nucleic acid (e.g., DNA or RNA) or nucleic acid mimetics (e.g., phosphorothionate mimetics) as are known in the art. The antisense molecule, ribozyme or RNAi molecule can be in a pharmaceutical composition that preferably comprises an excipient that enhances penetration of the antisense molecule, ribozyme or RNAi molecule into cells. The antisense molecule, ribozyme or RNAi can be expressed from a vector. Such vectors are known in the art.

In other embodiments, the agent reduces the activity of Aif-1. In an embodiment of the methods described herein, the agent is a small molecule of 2000 daltons or less. In an embodiment of the methods described herein, the agent is a small molecule of 1500 daltons or less. In an embodiment of the methods described herein, the agent is a small molecule of 1000 daltons or less. In an embodiment of the methods described herein, the agent is a small molecule of 800 daltons or less. In an embodiment of the methods described herein, the agent is a small molecule of either 2000, 1500, 1000, 800, 700, 600, 500 or 400 daltons or less. In an embodiment of the methods described herein, the agent is a small organic molecule. Drugs that reduce the activity or expression of Aif-1 include the anti-inflammatory drug sodium salicylate (38).

The agent can be an antibody or antibody fragment that reduces the activity of Aif-1. Preferably, the antibody or antibody fragment specifically binds to Aif-1. Antibody fragments include, but are not limited to, F(ab′)₂ and Fab′ fragments and single chain antibodies. F(ab′)₂ is an antigen binding fragment of an antibody molecule with deleted crystallizable fragment (Fc) region and preserved binding region. Fab′ is ½ of the F(ab′)₂ molecule possessing only ½ of the binding region. The term antibody is further meant to encompass polyclonal antibodies and monoclonal antibodies. Antibodies may be produced by techniques well known to those skilled in the art. Polyclonal antibody, for example, may be produced by immunizing a mouse, rabbit, or rat with purified Aif-1. Monoclonal antibody may then be produced by removing the spleen from the immunized mouse, and fusing the spleen cells with myeloma cells to form a hybridoma which, when grown in culture, will produce a monoclonal antibody. The antibody can be, e.g., any of an IgA, IgD, IgE, IgG, or IgM antibody. The IgA antibody can be, e.g., an IgA1 or an IgA2 antibody. The IgG antibody can be, e.g., an IgG1, IgG2, IgG2a, IgG2b, IgG3 or IgG4 antibody. A combination of any of these antibodies subtypes can also be used. The antibody can be a human antibody or a non-human antibody such as a goat antibody or a mouse antibody. Antibodies can be “humanized” using standard recombinant DNA techniques.

Aptamers are single stranded oligonucleotides or oligonucleotide analogs that bind to a particular target molecule, such as a protein. Thus, aptamers are the oligonucleotide analogy to antibodies. However, aptamers are smaller than antibodies. Their binding is highly dependent on the secondary structure formed by the aptamer oligonucleotide. Both RNA and single stranded DNA (or analog) aptamers can be used. Aptamers that bind to virtually any particular target can be selected using an iterative process called SELEX, which stands for Systematic Evolution of Ligands by EXponential enrichment.

Aif-1 can also be downregulated by agents that suppress expression of a disintegrin and metalloproteinase domain 3 (ADAM3) (38).

The agent can be administered to the subject in a pharmaceutical composition comprising a pharmaceutically acceptable carrier. Examples of acceptable pharmaceutical carriers include, but are not limited to, additive solution-3 (AS-3), saline, phosphate buffered saline, Ringer's solution, lactated Ringer's solution, Locke-Ringer's solution, Krebs Ringer's solution, Hartmann's balanced saline solution, and heparinized sodium citrate acid dextrose solution. The pharmaceutically acceptable carrier used can depend on the route of administration. The pharmaceutical composition can be formulated for administration by any method known in the art, including but not limited to, oral administration, parenteral administration, intravenous administration, transdermal administration, intramuscular administration, intranasal administration, and administration through an osmotic mini-pump. The compounds can be applied to the skin, for example, in compositions formulated as skin creams, or as sustained release formulations or patches.

The invention further provides a method for screening for an agent that treats multiple sclerosis in a subject, the method comprising determining whether or not the agent reduces expression and/or activity of Allograft inflammatory factor-1 (Aif-1), wherein an agent that reduces expression and/or activity of Aif-1 is a candidate for treating multiple sclerosis.

The agent can, for example, reduce Aif-1 expression and/or activity in lymphocytes, and/or in macrophages and/or microglia.

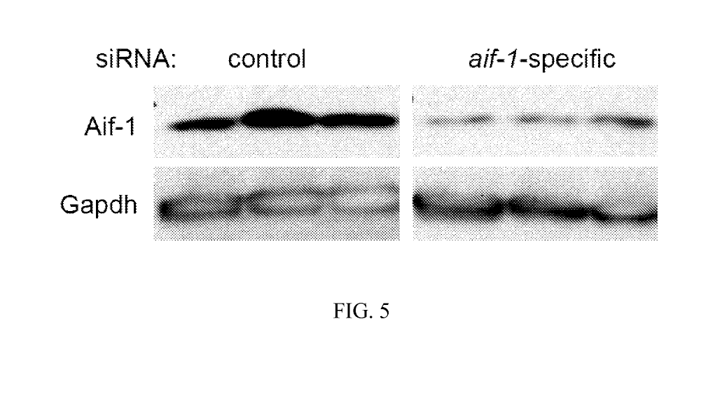

A cell culture-based assay can be used for initial screening. In that case, an enzyme-linked immunosorbent assay (ELISA) or Western blots can be used to assess Aif-1 levels in cellular lysates or in the cell culture medium. As an example, FIG. 5 shows a Western blot analysis of short interfering RNA (siRNA) knockdown of Aif-1 levels in cultured cells. In that example, RAW264.7 macrophages were transfected with non-targeting (control) or Aif-1-specific siRNAs. Total cellular lysates (in triplicate) were harvested after 48 h and Aif-1 protein levels were evaluated by Western analysis. Gapdh protein is shown as a loading control.

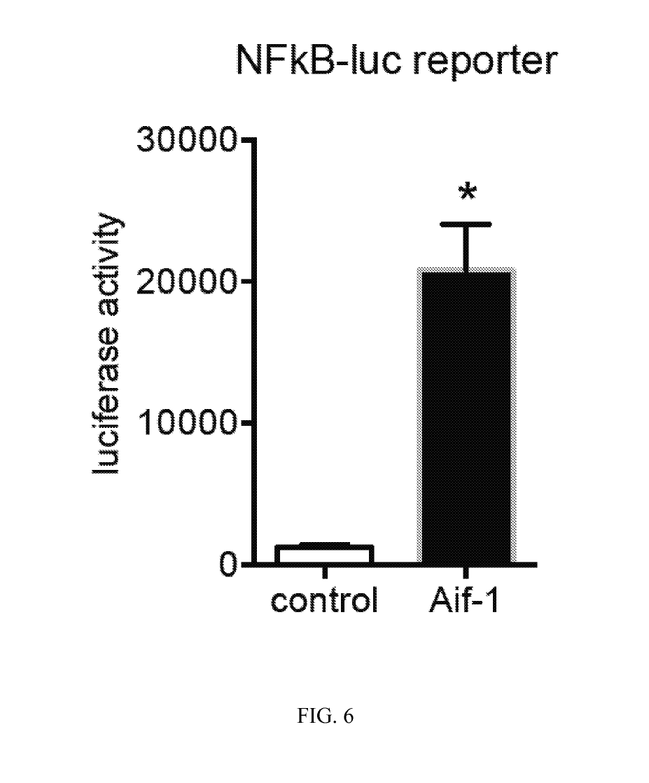

Aif-1 activity can be assessed, for example, using a Nuclear Factor kappa B (NFkB) reporter assay. Overexpression of Aif-1 in stimulated macrophages increases the activity of an NFkB-responsive reporter plasmid, in which luciferase activity is controlled by concatemerized NFkB DNA binding sites upstream of a minimal promoter and a gene encoding luciferase protein. As an example, FIG. 6 shows that Aif-1 potentiates NFkB activation. RAW264.7 macrophages were transfected with a control expression vector or Aif-1-encoding expression vector, and stimulated with interferon-gamma (100μ/ml) and LPS (5 ng/ml). After 8 hours, luciferase activity was assessed. An agent that inhibits Aif-1 activity would likewise limit the ability of exogenous or transfected Aif-1 to increase NFkB activity in such an assay.

For in vivo validation, a method to assess Aif-1 expression can be based on ELISA. There are a host of companies that now provide such assays that permit quantitative measurement of AiF-1 levels in animal (human, mouse, etc.) serum, plasma, tissue homogenates, cell culture supernatants and other biological fluids. Examples include USBiological, product #023250, Allograft Inflammatory Factor 1 (AIF1) BioAssay™ ELISA Kit (Human), and Biomatik, product #EKU02254, ELISA Kit for Allograft Inflammatory Factor 1 (AIF1), Homo sapiens (Human). Biomatik also has a CLIA kit for Aif-1, product #CKU72412. Since Aif-1 can be measured in human plasma (36), decreased circulating levels would correspond to lower plasma Aif-1 activity. Aif-1 activity could be compared before and after administration of an agent to the same subject, and/or after administration of an agent to a group of test subjects versus administration of a control to a group of control subjects.

In the methods described here, the agent can, for example, do one or more of reduce infiltration in the central nervous system by leukocytes and/or CD4⁺ T cells and/or microglia; reduce CD4⁺ T cell activation; reduce pro-inflammatory cytokine expression, such as, e.g., reduce expression of one or more of IL-6, IFN-γ, IL-12, and IL-2; and reduce demyelination.

In one embodiment of the methods described herein, the agent reduces expression of Aif-1. In one embodiment of the methods described herein, the agent reduces the activity of Aif-1.

In different embodiments, human Aif-1 protein can have, for example, the following amino acid sequence:

| (GenBank: AAD18087.1, SEQ ID NO: 26) | |
|---|---|
| 1 | msqtrdlqgg kafrllkaqq eerldeinkq flddpkyssd edlpsklegf kekymefdln |
| 61 | gngdidimsl krmleklgvp kthlelkkli gevssgsget fsypdflrmm lgkrsailkm |
| 121 | ilmyeekare kekptgppak kaiselp |
| or | |
| (GenBank: AAA92457.1, SEQ ID NO: 27) | |
| 1 | msqtrdlqgg kafgllkaqq eerldeinkq flhdpkyssd edlpsklegf kekymefdln |
| 61 | gngdidimsl krmleklgvp kthlelkkli gevssgsget fsypdflrmm lgkrsailkm |
| 121 | ilmyeekare rktntppsqe spi |
| or | |
| (NCBI Reference Sequence: NP_004838.1, SEQ ID NO: 28) | |
| 1 | mefdlngngd igekrvicgg rvvcrpkkte vsptcsiphd lgggppttvg grrmgmrkwe |
| 61 | rrervsppsp hphplppdim slkrmleklg vpkthlelkk ligevssgsg etfsypdflr |
| 121 | mmlgkrsail km |
| or | |
| (NCBI Reference Sequence: NP_116573.1, SEQ ID NO: 29) | |
| 1 | mefdlngngd idimslkrml eklgvpkthl elkkligevs sgsgetfsyp dflrmmlgkr |
| 61 | sailkmilmy eekarekekp tgppakkais elp. |

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

Experimental autoimmune encephalomyelitis (EAE), an animal model of human multiple sclerosis (MS), is mediated by myelin-specific auto-reactive CD4 T cells that cause inflammation and demyelination in the CNS. Allograft inflammatory factor-1 is induced in active MS and EAE lesions. The present study provides the first assessment of Aif-1 function in EAE pathogenesis. Mice lacking Aif-1 were used to evaluate the functional role of this molecule in EAE pathogenesis. The data demonstrate that deficiency of Aif-1 limits both the incidence and severity of EAE. At the tissue and cellular levels, these findings correspond to reduced cellular infiltration in the CNS, diminished demyelination, impaired expansion and activation of encephalitogenic CD4 T cells, and decreased expression of pro-inflammatory cytokines in the periphery; the consequences of potential impaired phagocytic activities appear to be functionally less important. These findings identify Aif-1 as a potent CD4 T cell-activating molecule in myelin oligodendrocyte glycoprotein (MOG)35-55-induced EAE and as a therapeutic target in multiple sclerosis.

### Materials and Methods

Animals.

Aif-1-deficient mice were generated through a homologous recombination gene targeting strategy (25). The targeted ail 1 allele was backcrossed onto the C57BL/6 strain for eight generations, and the corresponding knockout and wildtype (WT) littermates were bred in-house as homozygous or heterozygote lines in the barrier facility at the Albert Einstein College of Medicine. All experiments involving live animals were performed in accordance with protocols approved by the Albert Einstein College of Medicine IACUC.

Induction of EAE and Evaluation of Clinical Disease.

EAE was induced in mice as previously described (34). Briefly, 8-10 week old male mice were immunized subcutaneously in the lower dorsum with 300 μg of MOG35-55 peptide (MEVGWYRSPFSRVVHLYRNGK (SEQ ID NO:25); Celtek Bioscience) in a 200 μl emulsion of Incomplete Freunds Adjuvant (IFA) containing 5 mg/ml Mycobacterium tuberculosis H37RA (Difco Laboratories). Subsequent to immunization, the mice received intraperitoneal injections of pertussis toxin (500 ng, List Biological Laboratories) on the first day of sensitization and again after two days. The day after MOG immunization was designated Day 1. The EAE disease activity was scored as follows: 0, no symptoms; 1, floppy tail; 2, hind limb weakness; 3, hind limb paralysis; 4, fore limb and hind limb paralysis; and 5, death.

Histologic and Immunofluorescence Analysis of Spinal Cords.

For pathological analysis, EAE mice were anesthetized at the timepoints indicated and perfused with phosphate-buffered saline (PBS) via cardiac puncture. The spinal cord was flushed by hydrostatic pressure using PBS. The lumbar spinal cord was post-fixed overnight with 4% paraformaldehyde, and the tissues were paraffin-embedded. To assess infiltration, coronal sections (6 μm thickness) were stained with hematoxylin and eosin (H&E) and examined using Zeiss Axioskop II with MRC camera in the Einstein Analytic Imaging Facility (AIF). The extent of infiltration was quantified by measuring the infiltrated area of each individual spinal cord section normalized to total white matter area using Adobe Photoshop CS3 version 10 software, and expressed as the percentage of the total area.

To assess demyelination, paraffin-embedded spinal cord sections were deparaffinized and blocked with 10% donkey serum for 1 h at room temperature (RT) followed by antigen retrieval. The sections were incubated with anti-mouse myelin basic protein (MBP, 1/250 dilution, Covance) overnight at 4° C. and incubated with donkey anti-mouse Alexa 548 ( 1/250 dilution, Invitrogen) for 1 h at RT. The counterstained slides were mounted in aqueous mounting medium containing DAPI (Electron Microscopy Sciences) and examined using an Olympus IX 81 microscope with motorized stage and a Cooke Sensicam QE air-cooled charge-coupled device-bearing camera in the Einstein AIF. The extent of demyelination was quantified (Adobe Photoshop) by measuring the area of non MBP-stained white matter, normalized to total white matter area, and expressed as the percentage of the total area.

Isolation of Mononuclear Cells from CNS.

Spinal cords were perfused and flushed by hydrostatic pressure, and the recovered tissues were homogenized and digested with Collagenase A (2 mg/ml, Roche Diagnostics) in RPMI 1640 at 37° C. for 15 min. The digested tissues were filtered through a 100 μm cell strainer to obtain a single cell suspension and centrifuged at 500×g for 5 min. Cell pellets from 2 mice in each group were pooled, resuspended in 70% Percoll, overlaid with 30% Percoll, and centrifuged at 200×g for 15 min. The cell monolayer at the 70-30% interphase was collected and stained with various antibodies for flow cytometry, as described below.

Flow Cytometry Analysis.

At day 16 after EAE induction, spleen and peripheral lymph node cells were isolated, depleted of erythrocytes, blocked for Fc receptors RII/III with antibodies specific for CD16/CD32 (BD Pharmingen), and stained for surface markers with the following antibodies: anti-CD3-APC, anti-CD4-FITC, anti-CD8-PerCP, anti-B220-Pacific blue, anti-CD69-PE (BD Pharmingen), anti-CD45-Pacific blue (Biolegend) and anti-CD11b-APC (eBiosciences). The stained cells were analysed by FACS (LSRII, BD Biosciences), and the data were analyzed using FlowJo software (Tree Star).

Proliferation.

T cell and antigen-specific proliferation were assessed by stimulating splenocytes (4×10⁵ cells/well) from day 16 EAE with either α-CD3 (200 ng/ml) or MOG35-55 (20 μg/ml) for 72 h. Cells were incubated with [3H] thymidine (25 μCi/ml) for last 24 h, and incorporated radioactivity was measured using a β-counter and expressed as counts per minute (CPM).

T Cell Activation and Proliferation.

To evaluate T cell activation, splenocytes from naïve 10 week old wt and aif-I^{-/-} mice were isolated and enriched for CD4 T cells using an EasySep positive selection kit (Stemcell Technologies). CD4 T cells were seeded in a 12 well plate (3.5×10⁶/well) and stimulated with either DMSO or PMA (10 ng/ml) and ionomycin (500 ng/ml) in the presence of a protein transport inhibitor (GolgiPlug®, BD Biosciences, 1 μg/ml/10⁶ cells) for 5 h. Cells were harvested and subjected to intracellular staining with anti-IL-2-FITC (BD Pharmingen) and analysed by FACS (LSRII, BD Biosciences). Data were analyzed using FlowJo software (Tree Star). To assess T cell proliferation, splenocytes were stimulated with either α-CD3 (200 ng/ml) or MOG35-55 (20 μg/ml) for 72 h and proliferation was measured by adding [³H] thymidine (25 μCi/ml) for the last 24 h of the assay. Incorporated [³H] thymidine was measured using a β-counter and expressed as counts per minute (CPM).

Cytokine Expression Analysis.

Mononuclear cells were isolated from spleens of day 16 EAE-induced mice, and single cell suspensions were prepared in RPMI 1640 supplemented with 10% FBS, 1% penicillin-streptomycin, 1% L-glutamine, and β-mercaptoethanol. Splenocytes (4×10⁵ cells/well) were stimulated with MOG35-55 peptide (10 and 20 μg/ml) for 72 h. The levels of IL-6, IL-2, IFN-γ, and IL-12p40 in culture supernatants were determined by ELISA using antibodies to IL-6, IL-2, IFN-γ (BD Pharmingen), and IL-12p40 (R&D systems), respectively.

Real-Time Quantitative PCR.

Spleen tissues were homogenized with Trizol (Invitrogen), and total RNA was extracted using chloroform and precipitated with isopropanol. Synthesis of cDNA was performed using 2 μg of RNA using a reverse transcription system (Invitrogen). Real time PCR was performed using a Roche 480 light cycler using SYBR green quantitative master mix (Roche Applied Sciences). The relative expression of various cytokine and iNOS genes was determined in comparison to that of gapdh. Data were analyzed using the Pfaffl method (35). The following primers were used:

IL-6:
(SEQ ID NO: 1)
5′-GCTACCAAACTGGATATAATCAGGA-3′ (forward)
(SEQ ID NO: 2)
5′-CCAGGTAGCTATGGTACTCCAGAA-3′ (reverse)
IL-12p40:
(SEQ ID NO: 3)
5′-GATTCAGACTCCAGGGGACA-3′ (forward)
(SEQ ID NO: 4)
5′-TGGTTAGCTTCTGAGGACACATC-3′ (reverse)
IL-12p35:
(SEQ ID NO: 5)
5′-CCATCAGCAGATCATTCTAGACAA-3′ (forward)
(SEQ ID NO: 6)
5′-CGCCATTATGATTCAGAGACTG-3′ (reverse)
IL-2:
(SEQ ID NO: 7)
5′-GCTGTTGATGGACCTACAGGA-3′ (forward)
(SEQ ID NO: 8)
5′-TTCAATTCTGTGGCCTGCTT-3′ (reverse)
IL-4:
(SEQ ID NO: 9)
5′-CATCGGCATTTTGAACGAG-3′ (forward)
(SEQ ID NO: 10)
5′-CGAGCTCACTCTCTGTGGTG-3′ (reverse)
IFN-γ:
(SEQ ID NO: 11)
5′-ATCTGGAGGAACTGGCAAAA-3′ (forward)
(SEQ ID NO: 12)
5′-TTCAAGACTTCAAAGAGTCTGAGGTA-3′ (reverse)
TNF-α:
(SEQ ID NO: 13)
5′-TCTTCTCATTCCTGCTTGTGG-3′ (forward)
(SEQ ID NO: 14)
5′-GGTCTGGGCCATAGAACTGA-3′ (reverse)
IL-17:
(SEQ ID NO: 15)
5′-CAGGGAGAGCTTCATCTGTGT-3′ (forward)
(SEQ ID NO: 16)
5′-GCTGAGCTTTGAGGGATGAT-3′ (reverse)
IL-23p19:
(SEQ ID NO: 17)
5′-TCCCTACTAGGACTCAGCCAAC-3′ (forward)
(SEQ ID NO: 18)
5′-TGGGCATCTGTTGGGTCT-3′ (reverse)
iNOS:
(SEQ ID NO: 19)
5′-GGGCTGTCACGGAGATCA-3′ (forward)
(SEQ ID NO: 20)
5′-CCATGATGGTCACATTCTGC-3′ (reverse)
IL-10:
(SEQ ID NO: 21)
5′-CAGAGCCACATGCTCCTAGA-3′ (forward)
(SEQ ID NO: 22)
5′-GTCCAGCTGGTCCTTTGTTT-3′ (reverse)
IL-13:
(SEQ ID NO: 23)
5′-CCTCTGACCCTTAAGGAGCTTAT-3′ (forward)
(SEQ ID NO: 24)
5′-CGTTGCACAGGGGAGTCT-3′ (reverse).

Statistical Analysis.

Data are represented as mean±SEM. Two-tailed Student's t test, two-way ANOVA, and Mann-Whitney-U test were used to assess statistical significance. P-values <0.05 were considered statistically significant. Quantitative analyses were performed with Prism (GraphPad Software).

### Results and Discussion

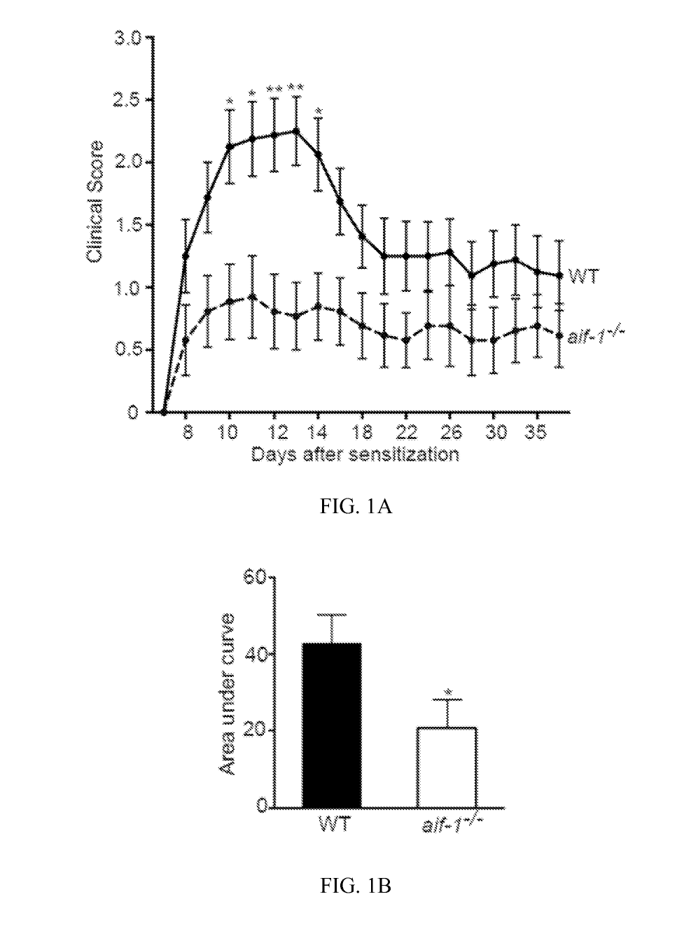

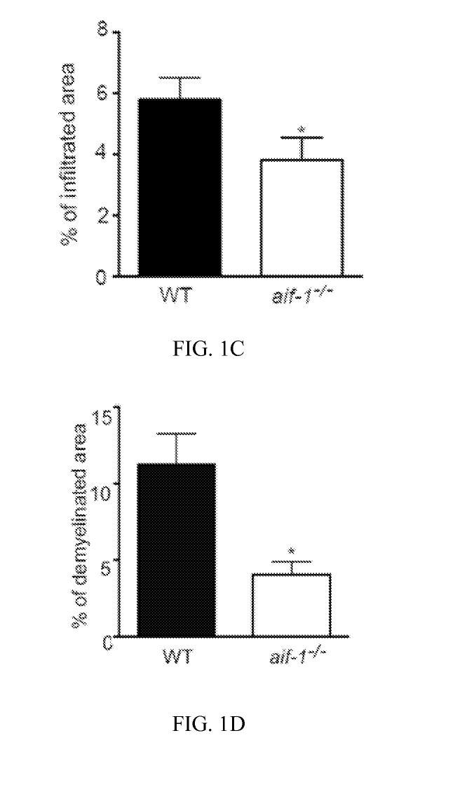

Mice lacking Aif-1 show lower incidence and reduced clinical severity of EAE. Aif-1 is induced in microglial cells in different stages of EAE in rat and mouse models (21, 22), However, the functional contribution of Aif-1 to EAE pathogenesis has been unknown. Accordingly, the role of Aif-1 was assessed in EAE development by sensitizing wild type (WT) and aif-1−/− mice with MOG35-55 to induce EAE. Aif-1−/− mice developed less severe EAE compared to WT mice (FIG. 1A), as reflected in reduced mean clinical scores throughout a 6-week evaluation period, and overall clinical expression of disease as quantified by measuring the area under the curve (AUC) throughout the study period (FIG. 1B). Moreover, aif-1−/− mice displayed reduced EAE incidence, maximum clinical score, and cumulative disease index (CDI). However, the timing of disease onset and the time to peak disease were similar in both groups (Table I).

Aif-1 Deficiency in Mice Decreases EAE-Associated CNS Leukocyte Infiltration and Demyelination.

EAE is initiated by leukocyte infiltration in the CNS (26). To determine if the neurological sparing observed in aif-1−/− mice is due to differences in leukocyte infiltration, H&E staining of spinal cord sections was performed from day 16 EAE-induced WT and aif-1−/− mice. These studies showed significantly reduced inflammatory cell infiltrates in aif-1−/− compared to WT mice; infiltrates were quantified (FIG. 1C), as described in methods. Demyelination and axonal damage occur as consequences of mononuclear cell recruitment (26), so the degree of demyelination in the spinal cord sections was assessed by staining for Myelin Basic Protein (MBP). Compared to WT, aif-1−/− mice displayed significantly less demyelination, as evidenced by preserved staining for MBP. Quantitative analysis also supported this observation (FIG. 1D). Overall, these findings show that immune cell infiltration and demyelination were both significantly decreased in the spinal cords of aif-1−/− mice, consistent with the decreased incidence and severity of disease.

Aif-1 Deficiency Reduces CNS Infiltration by CD4 T Cells.

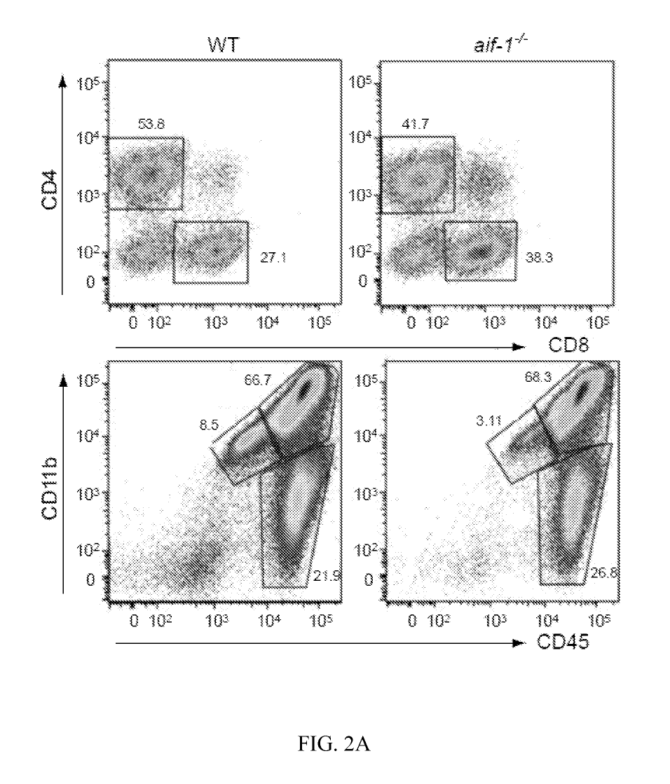

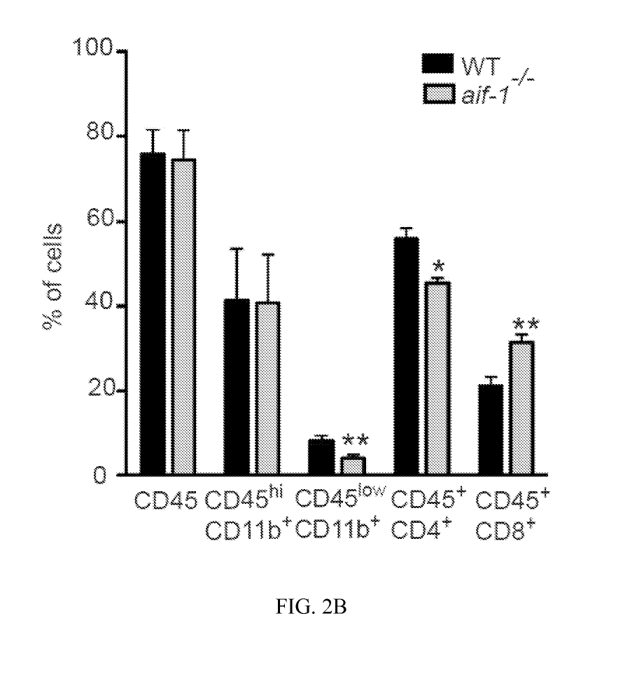

Genetic linkage studies describe strong association of MS with MHC class II alleles, and the presence of additional risk loci within the MHC remains a point of debate (27). Although auto-reactive CD4 T cells have generally been regarded as the major immune drivers of EAE/MS pathogenesis, recent failure of CD4 T cell-directed therapies plus observations of increased CD8 T cell numbers in MS plaques have led some workers to postulate an important disease-promoting role for CD8 T cells (28, 29). On the other hand, other investigators have shown a regulatory role for CD8 T cells in EAE (30). To determine how Aif-1 deficiency affected the composition of CNS leukocyte populations after EAE induction, inflammatory cells were profiled in spinal cords from both WT and aif-1−/− mice using FACS of mononuclear cells isolated from mice 16 days after immunization. Compared to WT, aif-1−/− spinal cords had fewer CD45lowCD11b+ microglia, fewer CD4 T cells, and more CD8 T cells. No differences were observed in CD45int-high CD11b+ activated microglia or infiltrated monocytes (FIGS. 2, A and B). This decrease in the microglial population could reflect an essential role for Aif-1 in microglial survival or repopulation (31). These possibilities were addressed by analyzing resident microglial populations (CD45^{low}CD11b⁺) in wt and aif-1^{-/-} naïve mice by FACS. No difference was found between the two groups (data not shown), which suggests that Aif-1 is not necessary for microglial survival or repopulation at baseline. Taken together, these data show that Aif-1 inactivation limits CNS CD4 T cell infiltration and demyelination in EAE, resulting in decreases in disease incidence and severity compared to WT mice.

Aif-1 Promotes Expansion and Activation of Encephalitogenic CD4 T Cells in the Periphery.

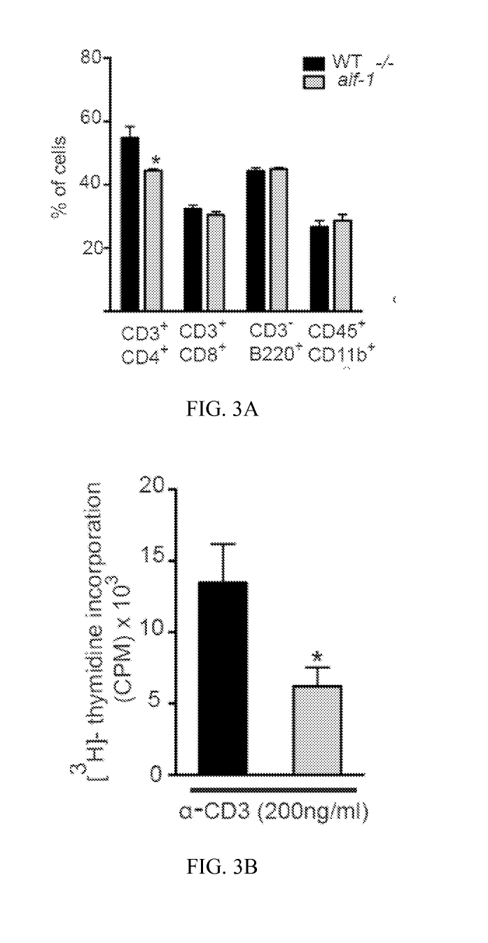

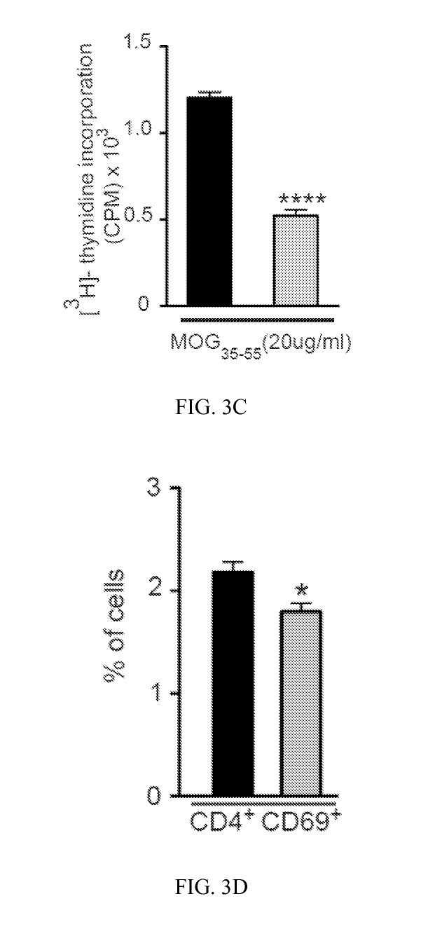

In EAE, myelin-specific T cells are activated in the periphery and migrate into the CNS followed by permeabilization of the blood brain barrier (32, 33). To investigate if the decrease in disease severity, CNS infiltration, and preserved myelin observed in Aif-1-deficient mice (FIGS. 1, 2) reflect differences in immune responses in the periphery, splenocytes and lymph node cells were isolated from day 16 EAE-sensitized mice and various immune subsets were assessed. Splenocytes from aif-1−/− mice had a significantly lower percentage of CD4 T cells (FIG. 3A) compared to WT mice. No significant differences were observed in the percentage of splenic B cells (B220+), CD8+ T cells, or monocytes (CD45+CD11b+) (FIG. 3A). Furthermore, there were no differences observed between aif-1−/− and WT mice in T cell subpopulations (CD4+, CD8+), B cells (CD3-B220+) and macrophages (CD45+CD11b+) in lymph node cells (data not shown).

It was investigated whether the lower percentage of CD4 T cells observed in MOG35-55 immunized Aif-1 deficient mice might reflect a developmental deficiency in T cell subsets. In various immune cell populations from naïve wt and aif-1−/− mice (splenocytes) and peripheral blood, and thymic T cells, FACS analysis failed to find significant genotype-dependent differences in T cell subsets (data not shown). These findings suggests that lack of Aif-1 does not affect baseline T cell development, but limits the ability of CD4 T cells to expand in response to specific immunization.

To evaluate further if lower CD4+ T cell numbers in Aif-1-deficient spleens are due to impaired proliferation, splenocytes from WT and aif-1−/− mice were challenged with either anti-CD3 or MOG35-55. With either the general T cell activator or the specific antigen rechallenge, cells from aif-1−/− mice proliferated less than WT control (FIGS. 3, B and C). On the other hand, activation of T cells from naïve wt and aif-1−/− mice by phorbol ester and ionomycin was equivalent (data not shown), which shows that the Aif-1 deficiency affects acquired but not basal T cell responsiveness. Collectively, these data suggest that Aif-1 promotes myelin-specific CD4 T cell expansion in the spleen, which in turn supports CD4 T cell infiltration and demyelination of the spinal cord in EAE. Because antigen stimulation also promotes immune cell recruitment to and activation in lymph nodes, the effect of Aif-1 deficiency on lymph node populations after MOG35-55 immunization was also determined; these studies showed no significant differences between wt and aif-1−/− mice in lymph node populations including T cell subsets, B cells, and monocytes (data not shown).

Aif-1 Deficiency Promotes Th1 to Th2 Bias in Spleen Via Reduced CD4 T Cell Activation.

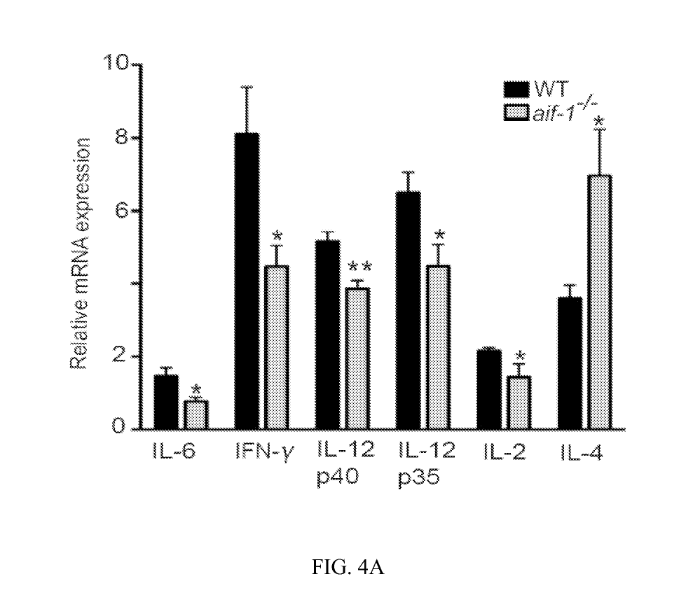

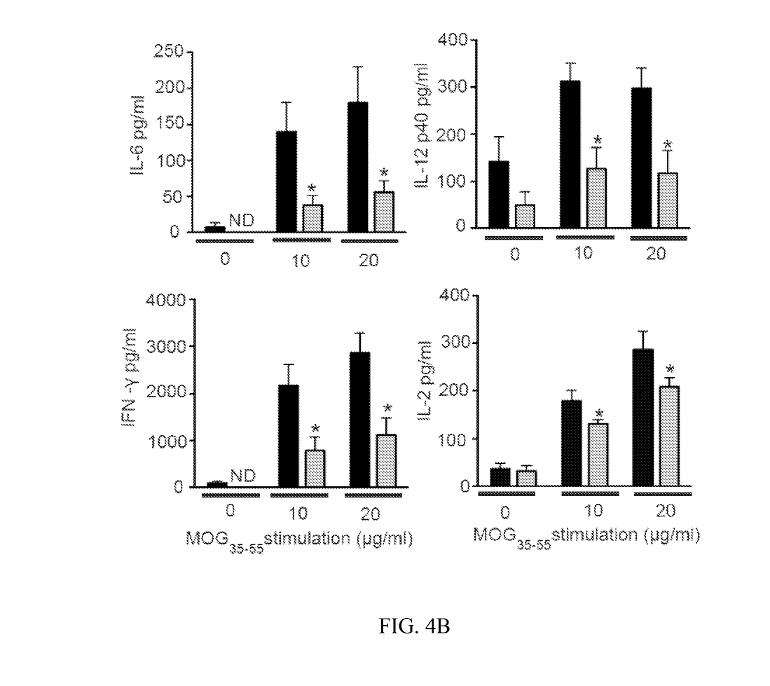

Auto reactive CD4 T cell activation and their associated proinflammatory cytokine milieu play important roles in the pathogenesis of EAE and MS. It was assessed whether lack of aif-1 affected B and T cell subset activation and cytokine production in day 16 EAE splenocytes from WT and aif-1−/− mice. Decreased CD4 T cell activation (CD4+CD69+, FIG. 3D) was observed in aif-1−/− mice compared to controls, but no differences were seen in activation of CD8 T and B cells (CD8+CD69+, B220+CD69+, data not shown). Furthermore, compared to WT controls, EAE-induced aif-1−/− samples showed significantly reduced expression of mRNAs encoding IL-6, IL-2, IL-12p35, IL-12p40, and IFN-γ but increased levels of IL-4 mRNA (FIG. 4A), consistent with the idea that deletion of Aif-1 promotes a Th1 to Th2 switch in the spleen. Trends toward decreased TNF-α and IL-13 and increased IL-10 levels were observed in aif-1−/− samples compared to WT, though these differences did not attain statistical significance (data not shown). Not all Th1 markers were affected, as no difference was found in expression of mRNAs encoding inducible NO synthase (iNOS), IL-17, and IL-23 between the two groups (data not shown). Consistent with these mRNA findings, antigen recall experiments showed significantly reduced levels of IL-6, IL-12p40, IL-2, and IFN-γ protein in supernatants of MOG35-55-stimulated splenocytes from aif-1−/− mice compared to WT mice (FIG. 4B), with no differences in the levels of TNF-α or IL-23 (not shown). Since no differences were found in expression of either IL-23 or IL-17 between wt and aif-1−/− samples, it was investigated if the Th1 to Th2 switch with loss of Aif-1 could be due to an increase in induced regulatory T (iTreg) cells (37). iTreg populations were assessed in splenocytes and lymph node cells from day 16 EAE mice by FACS; no significant differences were found between the groups (data not shown). Taken together, the data demonstrate that Aif-1 deficiency limits CD4 T cell activation and the proinflammatory nature of the cytokine milieu in MOG35-55-sensitized spleens, without affecting iTreg levels.

Although baseline peripheral, splenic, thymic lymphocyte and CNS microglial populations were similar in wt and aif-1−/− mice, the Aif-1-deficient mice had lower incidence and severity of induced disease. This corresponded to reduced CNS leukocyte infiltration and demyelination, and was associated with impaired expansion and activation of myelin-specific CD4 T cells and decreased pro-inflammatory cytokine production in the periphery. These findings suggest that Aif-1-dependent pro-inflammatory activities are dominant in this setting, while its phagocytotic and clearance functions are less critical.

Interestingly, the effect of Aif-1 deficiency on leukocyte populations after immunization was relatively modest, with a decrease of ˜10% in the number of both CD3+CD4+ and CD4+CD69+ T cells; on the other hand, proliferation of splenocytes lacking Aif-1 in response to either general anti-CD3 or specific MOG35-55 antigen challenge was reduced by ˜50%. This markedly impaired proliferative response was accompanied by a substantial reduction in several important Th1 cytokines, including IL-6, IFN-γ, IL-12, and IL-2, plus an increase in the Th2 cytokine IL-4, suggesting that loss of Aif-1 limits Th1− while enhancing Th2-type immune responses. No differences were observed in the iTreg cell population, nor in expression of markers of Th17 differentiation, including IL-23 p19 and IL-17.

In conclusion, the present study shows that lack of Aif-1 protects against the development of MOG35-55-induced EAE. This protection is characterized by reduced leukocyte infiltration and demyelination in CNS, and is associated with impaired expansion and activation of myelin-specific CD4 T cells and decreased pro-inflammatory cytokine production in the periphery.

TABLE I
Development of EAE in WT and aif-1^{-/-} mice

| | day of | days to peak | mean clinical | | | maximum | |
|---|---|---|---|---|---|---|---|
| genotype | onset | clinical disease | score | CDI (%) | incidence (%) | score | mortality |
| WT | 8.1 ± 0.61 | 10.1 ± 0.84 | 1.46 ± 0.13 | 28 ± 4.5 | 15/16 (94%) | 2.6 ± 0.26 | none |
| aif-1^{-/-} | 8.6 ± 1.3 | 10.6 ± 0.84 | 0.67 ± 0.05*** | 13 ± 4.5* | 8/13 (62%) | 1.5 ± 0.33* | none |

Day of onset corresponds to the second consecutive day in which an animal was scored 0.5 or higher. Days to peak clinical disease is the average of times until each animal received its highest score. Maximum score was calculated as the average of the highest clinical scores for each animal. Incidence is the fraction of animals with scores 0.5 or higher during the entire disease course. Cumulative disease index (CDI) is the sum of the daily EAE scores for each mouse for the entire duration of the experiment.
Data are represented as mean ± SEM..
*P < 0.05,
***P < 0.0001.

### REFERENCES

1. Frohman, E. M., M. K. Racke, and C. S. Raine. 2006. Multiple sclerosis—the plaque and its pathogenesis. The New England Journal of Medicine 354: 942-955.
2. Compston, A., and A. Coles. 2008. Multiple sclerosis. Lancet 372: 1502-1517.
3. Martin, R., and H. F. McFarland. 1995. Multiple sclerosis: are HLA class I molecules involved in disease pathogenesis? Ann Neurol 38: 137-139.
4. Wekerle, H., K. Kojima, J. Lannes-Vieira, H. Lassmann, and C. Linington. 1994. Animal models. Ann Neurol 36 Suppl: S47-53.
5. Miller, S. D., and W. J. Karpus. 1994. The immunopathogenesis and regulation of T-cell-mediated demyelinating diseases. Immunol Today 15: 356-361.
6. Langrish, C. L., Y. Chen, W. M. Blumenschein, J. Mattson, B. Basham, J. D. Sedgwick, T. McClanahan, R. A. Kastelein, and D. J. Cua. 2005. IL-23 drives a pathogenic T cell population that induces autoimmune inflammation. J Exp Med 201: 233-240.
7. Zhang, R., A. Tian, H. Zhang, Z. Zhou, H. Yu, and L. Chen. 2011. Amelioration of experimental autoimmune encephalomyelitis by beta-elemene treatment is associated with Th17 and Treg cell balance. J Mol Neurosci 44: 31-40.
8. Steinman, L., and S. S. Zamvil. 2005. Virtues and pitfalls of EAE for the development of therapies for multiple sclerosis. Trends Immunol 26: 565-571.
9. Domingues, H. S., M. Mues, H. Lassmann, H. Wekerle, and G. Krishnamoorthy. 2010. Functional and pathogenic differences of Th1 and Th17 cells in experimental autoimmune encephalomyelitis. PloS One 5: e15531.
10. Napoli, I., and H. Neumann. 2010. Protective effects of microglia in multiple sclerosis. Exp Neurol 225: 24-28.
11. Jiang, Z., J. X. Jiang, and G. X. Zhang. 2014. Macrophages: A double-edged sword in experimental autoimmune encephalomyelitis. Immunol letters 160: 17-22.
12. Iris, F. J., L. Bougueleret, S. Prieur, D. Caterina, G. Primas, V. Perrot, J. Jurka, P. Rodriguez-Tome, J. M. Claverie, J. Dausset, and et al. 1993. Dense Alu clustering and a potential new member of the NF kappa B family within a 90 kilobase HLA class III segment. Nature Gen 3: 137-145.
13. Deininger, M. H., R. Meyermann, and H. J. Schluesener. 2002. The allograft inflammatory factor-1 family of proteins. FEBS Letters 514: 115-121.
14. Autieri, M. V. 1996. cDNA cloning of human allograft inflammatory factor-1: tissue distribution, cytokine induction, and mRNA expression in injured rat carotid arteries. Biochem Biophys Res Comm 228: 29-37.
15. Hara, H., M. Ohta, K. Ohta, M. Nishimura, H. Obayashi, and T. Adachi. 1999. Isolation of two novel alternative splicing variants of allograft inflammatory factor-1. Biol Chem 380: 1333-1336.
16. Del Galdo, F., and S. A. Jimenez. 2007. T cells expressing allograft inflammatory factor 1 display increased chemotaxis and induce a profibrotic phenotype in normal fibroblasts in vitro. Arth Rheum 56: 3478-3488.
17. Kelemen, S. E., and M. V. Autieri. 2005. Expression of allograft inflammatory factor-1 in T lymphocytes: a role in T-lymphocyte activation and proliferative arteriopathies. Am J Pathol 167: 619-626.
18. Utans, U., W. C. Quist, B. M. McManus, J. E. Wilson, R. J. Arceci, A. F. Wallace, and M. E. Russell. 1996. Allograft inflammatory factory-1. A cytokine-responsive macrophage molecule expressed in transplanted human hearts. Transplantation 61: 1387-1392.
19. Zhao, Y. Y., D. J. Yan, and Z. W. Chen. 2013. Role of AIF-1 in the regulation of inflammatory activation and diverse disease processes. Cell Immunol 284: 75-83.
20. Lorente-Cebrian, S., P. Decaunes, E. Dungner, A. Bouloumie, P. Arner, and I. Dahlman 2013. Allograft inflammatory factor 1 (AIF-1) is a new human adipokine involved in adipose inflammation in obese women. BMC Endocrine disorders 13: 54.
21. Schluesener, H. J., K. Seid, J. Kretzschmar, and R. Meyermann. 1998. Allograft-inflammatory factor-1 in rat experimental autoimmune encephalomyelitis, neuritis, and uveitis: expression by activated macrophages and microglial cells. Glia 24: 244-251.
22. Lu, J., M. Kurejova, L. N. Wirotanseng, R. A. Linker, R. Kuner, and A. Tappe-Theodor. 2012. Pain in experimental autoimmune encephalitis: a comparative study between different mouse models. J Neuroinflamm 9: 233.
23. Watano, K., K. Iwabuchi, S. Fujii, N. Ishimori, S. Mitsuhashi, M. Ato, A. Kitabatake, and K. Onoe. 2001. Allograft inflammatory factor-1 augments production of interleukin-6, -10 and -12 by a mouse macrophage line. Immunology 104: 307-316.
24. Ohsawa, K., Y. Imai, H. Kanazawa, Y. Sasaki, and S. Kohsaka. 2000. Involvement of Iba1 in membrane ruffling and phagocytosis of macrophages/microglia. J Cell Sci 113 (Pt 17): 3073-3084.
25. Casimiro, I., P. Chinnasamy, and N. E. Sibinga. 2013. Genetic inactivation of the allograft inflammatory factor-1 locus. Genesis.
26. Steinman, L. 1999. Assessment of animal models for MS and demyelinating disease in the design of rational therapy. Neuron 24: 511-514.
27. Sawcer, S., M. Ban, M. Maranian, T. W. Yeo, A. Compston, A. Kirby, M. J. Daly, P. L. De Jager, E. Walsh, E. S. Lander, J. D. Rioux, D. A. Hafler, A. Ivinson, J. Rimmler, S. G. Gregory, S. Schmidt, M. A. Pericak-Vance, E. Akesson, J. Hillert, P. Datta, A. Oturai, L. P. Ryder, H. F. Harbo, A. Spurkland, K. M. Myhr, M. Laaksonen, D. Booth, R. Heard, G. Stewart, R. Lincoln, L. F. Barcellos, S. L. Hauser, J. R. Oksenberg, S. J. Kenealy, J. L. Haines, and C. International Multiple Sclerosis Genetics. 2005. A high-density screen for linkage in multiple sclerosis. Am J Hum Genet 77: 454-467.
28. Weiss, H. A., J. M. Millward, and T. Owens. 2007. CD8+ T cells in inflammatory demyelinating disease. J Neuroimmunol 191: 79-85.
29. Friese, M. A., and L. Fugger. 2009. Pathogenic CD8(+) T cells in multiple sclerosis. Ann Neurol 66: 132-141.
30. Chen, M. L., B. S. Yan, D. Kozoriz, and H. L. Weiner. 2009. Novel CD8+ Treg suppress EAE by TGF-beta- and IFN-gamma-dependent mechanisms. Eur J Immunol 39: 3423-3435.
31. Derecki, N. C., J. C. Cronk, Z. Lu, E. Xu, S. B. Abbott, P. G. Guyenet, and J. Kipnis. 2012. Wild-type microglia arrest pathology in a mouse model of Rett syndrome. Nature 484: 105-109.
32. Furtado, G. C., M. C. Marcondes, J. A. Latkowski, J. Tsai, A. Wensky, and J. J. Lafaille. 2008. Swift entry of myelin-specific T lymphocytes into the central nervous system in spontaneous autoimmune encephalomyelitis. J Immunol 181: 4648-4655.
33. O'Connor, R. A., C. T. Prendergast, C. A. Sabatos, C. W. Lau, M. D. Leech, D. C. Wraith, and S. M. Anderton. 2008. Cutting edge: Th1 cells facilitate the entry of Th17 cells to the central nervous system during experimental autoimmune encephalomyelitis. J Immunol 181: 3750-3754.
34. Lutz, S. E., C. S. Raine, and C. F. Brosnan. 2012. Loss of astrocyte connexins 43 and 30 does not significantly alter susceptibility or severity of acute experimental autoimmune encephalomyelitis in mice. J Neuroimmunol 245: 8-14.
35. Pfaffl, M. W. 2001. A new mathematical model for relative quantification in real-time RT-PCR. Nucleic Acids Res 29: e45.
36. Fukui M, Tanaka M, Toda H, Asano M, Yamazaki M, Hasegawa G, Imai S, Fujinami A, Ohta M, Nakamura N. The serum concentration of allograft inflammatory factor-1 is correlated with metabolic parameters in healthy subjects. Metabolism. 2012 July; 61(7):1021-5.
37. Korn, T., M. Mitsdoerffer, and V. K. Kuchroo. 2010 Immunological basis for the development of tissue inflammation and organ-specific autoimmunity in animal models of multiple sclerosis. Results Probl Cell Differ. 51: 43-74.
38. Yang Z F, Ho D W, Lau C K, Lam C T, Lum C T, Poon R T, Fan S T. Allograft inflammatory factor-1 (AIF-1) is crucial for the survival and pro-inflammatory activity of macrophages. Int Immunol. 2005 November; 17(11):1391-7. Epub 2005 Sep. 12.

What is claimed is:

1. A method of treating multiple sclerosis in a subject comprising administering to the subject an agent in an amount effective to reduce expression and/or activity of Allograft inflammatory factor-1 (Aif-1) in a subject.

2. The method of claim 1, wherein Aif-1 expression and/or activity is reduced in lymphocytes.

3. The method of claim 1, wherein Aif-1 expression and/or activity is reduced in macrophages and/or microglia.

4. The method of claim 1, wherein the agent is an antisense molecule, a ribozyme, or an RNA interference (RNAi) molecule, and the agent reduces expression of Aif-1.

5. The method of claim 1, wherein the agent is a small molecule, an antibody, an antibody fragment, or an aptamer, and the agent reduces the activity of Aif-1.

6. The method of claim 1, wherein the agent does one or more of reduce infiltration in the central nervous system by leukocytes and/or CD4⁺ T cells and/or microglia; reduce CD4⁺ T cell activation; reduce pro-inflammatory cytokine expression, and reduce demyelination.

7. The method of claim 1, wherein the agent reduces expression of Aif-1.

8. The method of claim 1, wherein the agent reduces the activity of Aif-1.

9. A method for screening for an agent that treats multiple sclerosis in a subject, the method comprising determining whether or not the agent reduces expression and/or activity of Allograft inflammatory factor-1 (Aif-1), wherein an agent that reduces expression and/or activity of Aif-1 is a candidate for treating multiple sclerosis.

10. The method of claim 9, wherein the agent reduces Aif-1 expression and/or activity in lymphocytes.

11. The method of claim 9, wherein the agent reduces Aif-1 expression and/or activity in macrophages and/or microglia.

12. The method of claim 9, wherein the method is carried out using a cell culture based assay that uses an enzyme-linked immunosorbent assay (ELISA) or Western blots to assess Aif-1 levels in cellular lysates or in the cell culture medium.

13. The method of claim 9, wherein Aif-1 activity is assessed using a Nuclear Factor kappa B (NFkB) reporter assay.

14. The method of claim 9, wherein AiF-1 levels are assessed in serum, plasma, tissue homogenates, cell culture supernatants or another biological fluid.

15. The method of claim 9, wherein the agent does one or more of reduce infiltration in the central nervous system by leukocytes and/or CD4⁺ T cells and/or microglia; reduce CD4⁺ T cell activation; reduce pro-inflammatory cytokine expression, and reduce demyelination.

16. The method of claim 9, wherein the agent reduces expression of Aif-1.

17. The method of claim 9, wherein the agent reduces the activity of Aif-1.
